(19) United States
(12) Patent Application Publication
(10) Pub. No.: US 2017/0367809 A1
(43) Pub. Date: Dec. 28, 2017

(54) HUMAN IMPLANTABLE TISSUE EXPANDERS

(71) Applicant: IMPLITE LTD., Petach Tikva (IL)

(72) Inventor: Ami GLICKSMAN, Petach Tikva (IL)

(21) Appl. No.: 15/540,063

(22) PCT Filed: Dec. 28, 2015
(86) PCT No.: PCT/IL2015/051257

§ 371 Date: Jun. 27, 2017

Related U.S. Application Data
(60) Provisional application No. 62/097,084, filed on Dec. 28, 2014.

(51) Int. Cl. A61F 2/02; A61F 2/12; A61F 2/08

(52) U.S. Cl. CPC A61F 2/02; A61F 2/12; A61F 2/08; A61F 2210/0076; A61F 2002/0894

(57) ABSTRACT

Human implantable tissue expanders are provided, such as breast tissue expanders, that comprise an inner foam filling enclosed within a liquid-filled compartment and further within a sealing shell comprising a substantially non-stretchable resilient expansion restricting layer. The substantially non-stretchable resilient expansion restricting layer is configured to retain a shape and/or volume of said foam filling upon changes of ambient pressure and/or temperature.

### FIELD OF THE INVENTION

The present invention relates to human implantable tissue expanders, suitable, inter alia, for augmentation or reconstruction of breast, pectorals, calf muscles and other soft tissue defects.

### BACKGROUND OF THE INVENTION

Soft-tissue implants are used in various locations in the human body. The most common use is for reconstructing or improving the normal body contour or augmenting the female breast. The most common breast prostheses generally include a flexible elastomeric shell or envelope, typically made of silicone, which is filled with a soft gel, mainly silicone gel, a saline solution or a combination of both.

U.S. Pat. No. 3,683,424 discloses a compound prosthesis that has an elastic sack or envelope which contains an open-cell foam core and a quantity of a liquid in the cells of the core. The envelope has a flexible tube for adding the liquid at time of implantation so the size of the implant can be adjusted as desired.

U.S. Pat. No. 4,298,998 discloses a breast prosthesis claiming to overcome the tightness and contracture of the fibrous capsule which forms around an existing prosthesis. The construction of the prosthesis causes the capsule to form at a predetermined, controlled distance from the surface thereof. This prosthesis is constructed with a first phase or outer temporary component and a second phase or inner permanent component. The inner component is a container or sac of a flexible, non-absorbable material filled with a fluid or gel filler material. The temporary outer component is an outer container or cover of a material which is absorbable under the conditions of use, and an inert filler material, preferably an absorbable, biologically acceptable liquid, e.g. saline solution, filling the space between the inner and outer components.

U.S. Pat. No. 4,650,487 discloses a surgically implantable, multi-lumen, high profile mammary implant which includes a first, flexible, elastic lumen at least partly filled with a soft gel material and having a front wall approximating the shape of a human breast and a second, firmer, flexible lumen within the first lumen and connected thereto solely at the rear wall of the first lumen. A third lumen, preferably inflatable, surrounds the first lumen and is inflated with saline solution.

U.S. Pat. No. 5,236,454 discloses an implantable stacked breast prosthesis comprising two or more separate chambers stacked on each other, and fastened together eccentrically, so as to give a normal contour to the reconstructed or augmented breast and to prevent slippage of the chambers. At least one of the chambers is collapsed and may be variably filled with liquid.

U.S. Pat. No. 5,358,521 discloses a multi-layer prosthesis that simulates tissue tactility by structuring the plurality of layers of material making up the prosthesis to include lubricant coating between the layers. It is the plurality of layers and the lubricity of their movement which contributes greatly to the tactile simulation of human tissue. Present in the prosthesis is a ballast lumen which moves freely and contributes mass and motility to the prosthesis.

U.S. Pat. No. 5,376,117 discloses breast prostheses for subcutaneous implantation for breast augmentation. The prostheses include an outer shell having a smooth non-porous outer envelope and a non-woven porous outer layer affixed to the envelope.

U.S. Pat. No. 5,437,824 discloses a breast prosthesis for implantation beneath the skin. In one preferred embodiment the prosthesis has an outer elastic shell which encloses a biocompatible fluid and a silicone foam insert of unitary construction having the shape and approximate consistency and tactility of breast tissue. The foam insert occupies substantially the entire volume enclosed by the shell of the implantable prosthesis and consists of a foam body that is molded to the shape of the breast. In another preferred embodiment, only a portion of the volume enclosed by the cell is occupied by the foam insert. In yet another embodiment, a foam insert comprising an open-cell and closed-cell foam body may be directly implanted beneath the skin for breast augmentation or reconstruction without a shell.

U.S. Pat. No. 5,824,081 discloses a tissue implant having visco-elastic characteristics which simulate the natural tissue that is intended to be augmented or replaced. The implant is comprised of a shell or envelope enclosing a compound foam body and a fluid filler material.

U.S. Pat. No. 6,187,043 discloses an implant and coverings for an implant for use in the human body. Coverings for implants are constructed to present a biocompatible surface to the body and to provide a textured surface which serves to disorganize scar tissue which forms around the implant.

U.S. Pat. No. 6,875,233 discloses a hinging breast implant capable of being variably sized and that includes an exterior shell and an inner bladder. The exterior shell is typically a bellows having a plurality of pleats so that the outer size of the implant is variable so that different sizes and shapes can be obtained. The inner bladder can be filled with a suitable filling material, liquid, gas or solid. As the bladder is filled, the exterior shell expands in a manner that creates a lifting effect and a ballooning effect.

U.S. Pat. No. 8,236,054 discloses an implantable soft tissue prosthesis comprising a hollow shell formed of a flexible elastomeric envelope, the shell having an inner volume and an exterior surface, when the inner volume is filled with an elastomeric silicone tubing that is preshaped conforming to the inner volume of the shell, the prosthesis being adapted to be surgically implanted in a human breast.

US 2002/0038147 discloses an improved permanently implantable breast tissue prosthesis comprising angularly and immutably attached base and dome envelopes wherein the base envelope is of a substantially triangular shape and the dome envelope is of a substantially discoid shape, each envelope having a shell defining an inner fluid containable chamber and an outer textured surface to be in direct contact with breast tissue and a valve formed as a part of a wall in base and dome envelopes, the valve facilitating the introduction, containment or removal of fluid within the containable chamber of each envelope.

US 2004/0162613 discloses a cosmetic and reconstructive prosthesis containing a rupture indicator, which includes an external envelope of medical grade elastomer containing a fluid material and a biologically compatible chemical indicator for indicating rupture of the prosthesis, and an internal envelope of medical grade elastomer disposed within the external envelope, the internal envelope containing an implant filling material.

WO 2006/114786 discloses a reduced weight implantable prosthesis, including an outer surface shell for encapsulating the prosthesis, a gel mixture comprising a mixture of cohesive gel and micro-spheres for filling the shell, and one or more inner volumes internal to the shell which do not contain the gel mixture.

WO 2007/000756, to the inventor of the present invention, discloses, inter alia, a human implantable tissue expander comprising a flexible enclosure for at least one material having at least one fluid flow characteristic; and a flexible and resilient skeleton associated with said flexible enclosure and being operative to maintain said flexible enclosure in a predetermined three-dimensional configuration generally independently of its orientation relative to gravitational acceleration.

WO 2008/081439, to the inventor of the present invention and others, discloses, inter alia, an implantable tissue expander including an internal skeletal element extending between a base surface and an outer surface and including at least one plurality of elongate cells extending along mutually generally parallel axes from the base surface to the outer surface and being defined by elongate cell walls formed of a resilient material; and a sealed enclosure, sealing the internal skeletal element and adapted for preventing body fluids from filling the plurality of elongate cells.

WO 2010/049926, to the inventor of the present invention, discloses, inter alia, a reconstructive breast prosthesis suitable for implantation into a void in a breast following a lumpectomy procedure in which a body of tissue is excised from the breast, the reconstructive breast prosthesis including an implant body at least generally configured to assume an implant shape corresponding to the shape of the body of tissue excised from the breast and an implant shape retaining structure adapted to maintain the implant body in the implant shape, the reconstructive breast prosthesis having an overall density which is less than the density of the body of tissue excised from the breast.

WO 2014/118773, to the inventor of the present invention, discloses human implantable tissue expanders that comprise an inner foam filling enclosed within a substantially non-stretchable resilient expansion restricting layer configured to retain a shape and/or volume of said foam filling upon changes of ambient pressure and/or temperature, and an outer shell comprising one or more layers formed of a resilient material.

There still remains a need for improved implantable tissue expanders.

### SUMMARY OF THE INVENTION

The present invention provides, according to some embodiments, human implantable tissue expanders comprising an inner foam filling made of a single foam element or multiple foam elements, enclosed within a liquid-filled compartment, such as silicone gel, saline or any other suitable liquid, and further within a shell made of one or more layers. The shell comprises a substantially non-stretchable resilient expansion restricting layer configured to retain a fixed surface area of said foam filling upon changes of ambient pressure, temperature or both. The inner foam filling of the implantable tissue expanders disclosed herein fills most of the volume of the implant, e.g., 60-95% of the total volume, thereby providing significant reduction of implant weight compared to known implants. The implantable tissue expanders disclosed herein are also advantageous for being soft, pliable and characterized by improved tactility.

In some embodiments, the shell comprising the substantially non-stretchable resilient expansion restricting layer is an outer shell enclosing both the inner foam filling and liquid-filled compartment. According to these embodiments, the implant may further comprise an additional, internal shell between the inner foam filling and liquid-filled compartment, enclosing the foam filling. The internal shell may be stretchable or may have an embedded expansion restricting layer like the outer shell, defining a fixed surface area to the inner foam filling. The latter results in an implant comprising two expansion restricting layers.

In other embodiments, the shell comprising the substantially non-stretchable resilient expansion restricting layer is an internal shell enclosing only the foam filling, such that the liquid-filled compartment surrounds both the foam filling and the shell. According to these embodiments, the implant further comprises an additional shell, an outer shell, enclosing the entire implant. In some embodiments, the additional outer shell is a flexible, stretchable shell. In other embodiments, the additional outer shell is also an expansion-restricting shell, like the inner shell. The latter results in an implant comprising two expansion restricting layers.

According to one aspect, there is provided a human implantable tissue expander comprising: an inner foam filling; a liquid-filled compartment enclosing the inner foam filling; and a shell comprising a substantially non-stretchable resilient expansion restricting layer configured to retain a fixed surface area of the foam filling upon changes of ambient pressure, temperature or both, wherein the inner foam filling constitutes at least 60% of the total volume of the tissue expander.

In some embodiments, the inner foam filling constitutes at least 75% of the total volume of the tissue expander. In some embodiments, the inner foam filling constitutes at least 95% of the total volume of the tissue expander.

In some embodiments, the inner foam filling comprises a single foam element. In other embodiments, the inner foam filling comprises plurality of foam elements, namely, at least two foam elements.

In some embodiments, the inner foam filling comprises closed-cell foam. In some embodiments, the closed-cell foam is silicone foam. In some embodiments, the closed-cell foam comprises closed-cell foam beads.

In some embodiments, the liquid is silicone gel. In other embodiments, the liquid is saline. The liquid may be any liquid known in the art that is suitable for use in implantable devices.

In some embodiments, the shell comprising a substantially non-stretchable expansion restricting layer is an outer shell enclosing the inner foam filling and the liquid-filled compartment. In some embodiments, such tissue expander further comprises an additional shell comprising a substantially non-stretchable expansion restricting layer, wherein the additional shell is an inner shell between the liquid-filled compartment and the inner foam filling, enclosing the inner foam filling.

In some embodiments, the tissue expander further comprises a shell comprising a stretchable layer formed of a resilient material.

In some embodiments, the shell comprising a stretchable layer formed of a resilient material is an outer shell enclosing the inner foam filling and the liquid-filled compartment, and the shell comprising a substantially non-stretchable resilient expansion restricting layer is an inner shell between the liquid-filled compartment and the inner foam filling, enclosing the inner foam filling.

In other embodiments, the shell comprising a stretchable layer formed of a resilient material is an inner shell between the liquid-filled compartment and the inner foam filling, enclosing the inner foam filling, and the shell comprising a substantially non-stretchable resilient expansion restricting layer is an outer shell enclosing the inner foam filling and the liquid-filled compartment.

In some embodiments, the tissue expander further comprises an outer layer of closed-cell foam.

In some embodiments, the tissue expander further comprises a code identifier on a surface thereof or embedded therein, for non-invasively identifying said tissue expander when implanted in a subject. The code identifier may be of a material resembling the mechanical properties of the shell, thus not changing the mechanical properties of the shell. In some embodiments, the code identifier is placed within any of the compartments of the tissue expander, or is partially embedded in a shell and partially protruding from the shell. The code identifier is made of a material having mechanical properties that do not damage the integrity of any shell or other components of the tissue expander.

These and further aspects and features of the present invention will become apparent from the figures, detailed description and claims which follow.

### BRIEF DESCRIPTION OF THE FIGURES

FIG. 1 to FIG. 6 are cross sectional illustrations of tissue expanders according to various embodiments of the present invention.

### DETAILED DESCRIPTION OF THE INVENTION

The present invention is directed to human implantable tissue expanders.

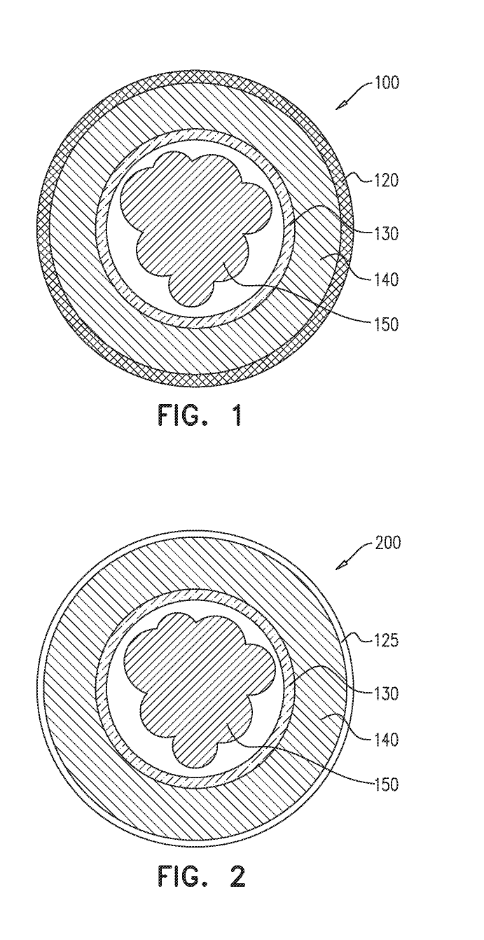

FIG. 1 illustrates a cross-sectional top view of a tissue expander 100 (used herein interchangeably with “implant”) according to some embodiments of the present invention, suitable, for example, for breast augmentation and/or reconstruction. In the embodiment illustrated in FIG. 1, the implant comprises an outer shell 120 comprising a substantially non-stretchable resilient expansion restricting layer, a liquid filled compartment 140, an inner shell 130 and an inner foam filling 150. Inner shell 130 is between liquid-filled compartment 140 and inner foam filling 150, enclosing inner foam filling 150. Inner shell 130 comprises a substantially non-stretchable resilient expansion restricting layer. In the illustrated embodiment, inner foam filling 150 is a single element of closed-cell foam that substantially fills inner shell 130. According to this embodiment, the closed-cell foam compartment substantially cannot expand, and both liquid-filled and foam compartments maintain a substantially fixed surface area. The liquid-filled compartment typically contains silicone gel or saline. The liquid-filled compartment is typically particle-fee.

An inner foam filling according to embodiments of the present invention constitutes at least about 60% of the total volume of the implant, for example at least 65%, at least 70%, at least 75%, at least 80%, at least 90%, and up to 95% of the total volume of the implant. Each possibility represents a separate embodiment of the present invention.

In some embodiments, the inner foam filling constitutes between 60 to 95% of the total volume of the implant. For example, the inner foam filling may constitute 60%-90% of the total volume, 65%-95% of the total volume, or 70%-90% of the total volume of the implant. Each possibility represents a separate embodiment of the present invention.

For example, for implants having a total volume of 250 cc, 300 cc and 350 cc, the volume of the foam filling when constituting 60% of the total volume is 150 cc, 180 cc and 210 cc, respectively. For implants having a total volume of 250 cc, 300 cc and 350 cc and filled with a foam filling constituting 90% of the total volume, the volume of the foam filling is 225 cc, 270 cc and 315 cc, respectively.

The volume ratio between the inner core containing the foam and the entire implant may be defined by a manufacturer according to the desired reduction in implant weight.

In some embodiments, the inner foam filling constitutes a single body (single element) of closed-cell foam, and in other embodiments the inner foam filling may constitute a plurality of closed-cell foam bodies (elements). The closed-cell foam bodies may be similar in shape or different in shape, such as randomly shaped foam bodies. The closed-cell foam bodies may also be manufactured in shapes that match each other to construct a predefined overall shape of the foam filling, according to a desired design.

An implant according to embodiments of the present invention is preferably resiliently deformable and partially compressible, and can be deformed or compressed to a deformed, compressed shape in which it has reduced dimensions, thereby permitting insertion of the implant through an aperture in a cutaneous layer when the implant is in the deformed, compressed shape, and allowing the implant, by virtue of its resiliency and ability to decompress, to regain its three dimensional shape when placed at a desired location within the body, for augmentation or reconstruction of a desired three dimensional shape of a body portion.

As used herein, the phrases “substantially non-stretchable expansion restricting layer”, “substantially non-expandable expansion restricting layer” or simply “expansion restricting layer”, refers according to some embodiments, to a layer, such as a mesh, that does not stretch or expand, (for example, elongate) in any direction to more than about 10% relative to its initial surface area under pressure changes of 0.5 atmosphere (for example, a pressure decrease from 1 atmosphere to 0.5 or 0.7 atmosphere). Preferably the expansion restricting layer does not stretch or expand to more than about 1-5% relative to its initial surface area under pressure changes of 0.5 atmosphere. According to some embodiments, the phrases “substantially non-stretchable expansion restricting layer”, “substantially non-expandable expansion restricting layer” or “expansion restricting layer”, refers to a layer, such as a mesh, that does not allow an increase of the volume enclosed within said layer to more than about 15% (preferably up to about) relative to the initial volume under pressure changes of 0.5 atmosphere. More preferably the expansion restricting layer does not stretch or expand at all under pressure changes of 0.5 atmosphere.

The expansion restricting layer defines a fixed surface area of the implant, preventing the expansion of the gas in the foam filling (and also air that may be entrapped between the foam and surrounding shell) during pressure decrease. According to some embodiments, the term “fixed” surface area may refer to a constant or substantially constant surface area, and indicates a surface area that does not change to more than about 1-5%.

As used herein, an “external shell” or an “outer shell” refers to a shell that is configured to contact surrounding body tissue upon implantation of the implant. This is in contrast to an “inner shell” or an “internal shell”, which refers to an internal component within the implant that is not configured to contact a body tissue.

As used herein, the term “about”, when referring to a measurable value, is meant to encompass variations of +/−10%, more preferably +/−5%, even more preferably +/−1%, and still more preferably +/−0.1% from the specified value.

The expansion restricting layer according to embodiments of the present invention is typically formed of a biocompatible material, such as polyester, polyethylene, polyamide, Gortex®, cellophane, aluminum foil or other materials known in the art as suitable for use for implantation in the human body.

The expansion restricting layer may be a woven fabric, a non-woven fabric, a knitted fabric or a sheet of material or a combination of such. The expansion restricting layer may be formed of two substantially non-expandable sheets joined together. The expansion restricting layer may be meshed. A knitted or woven layer may be characterized by the thickness of the layer being uniform or varied, and also by varied or uniform pore size, thread thickness and type of threads. The expansion restricting layer may be formed of a single piece, or multiple pieces or strands of material in any suitable manner, including, for example, weaving, injection molding, extruding, winding or wrapping. The expansion restricting layer may be closed to create a sealed enclosure by sewing, ultrasonic welding, gluing or other techniques known in the art.

In some embodiments, the expansion restricting layer is pre-formed, the foam filling is inserted inside the preformed expansion restricting layer, and the edges of the expansion restricting layer are then sealed to form a sealed expansion restricting layer enclosing the foam filling. In other embodiments, the expansion restricting layer is formed as an outer layer around the foam filling.

The expansion restricting layer has typically lower elongation capability and higher tensile strength capability compared to other layers/enclosures that constitute the implant according to embodiments of the present invention. In some embodiments, the expansion restricting layer is composed of a plurality of layers. For example, a mesh may compose a plurality of mesh layers.

The foam filling according to some embodiments of the present invention is typically a matrix characterized by a closed-cell structure filled with gas, for example, air-filled foam. In some embodiments, the foam comprises closed-cell foam beads. In other embodiments, the foam comprises a bulk structure having gas bubbles trapped inside it. In some typical embodiments, the foam is silicone foam. A particular example of silicone foam that can be used to produce a foam filling in accordance with the present invention is a two-part, room-temperature curing silicone foam such as MED-2310 (by Nusil Technology).

The foam filling can be produced by methods known in the art, for example, by mixing at room temperature two different biocompatible polymers, e.g. two types of silicone, that release gas (e.g., hydrogen, oxygen or ammonia) in an exothermic reaction upon mixing thereof. The generated gas is trapped within the silicone and generates closed-cell foam upon curing, meaning that each pocket of gas is completely surrounded by solid material. The gas is replaced spontaneously by air until partial gas pressure equilibrium is reached. Part of the outer layer of the foam may include open cells. Additionally, in order to change the consistency of the foam filling, the cured foam, either as a single element (single lump) or as several elements (lumps) of foam, may undergo pressure modification, e.g. weight milling that causes transformation of some of the closed cells into open cells, thus softening the consistency of the foam body. The density of the foam filling when filled with gas is generally less than about 0.5 gram per cubic centimeter and preferably less than about 0.3 gram per cubic centimeter. Pore size and number of cells per unit volume are typically defined by manufacturing parameters, such as the curing temperature and ambient pressure, and can vary according to the desired weight and consistency of the foam filling, as known in the art.

The foam filling has a defined shape that typically corresponds to its intended location within the body. The foam filling can be manufactured by molding, cutting partial volumes from a larger foam lump and joining them together, or extrusion. For example, a foam filling can be prepared by mixing two parts of uncured silicone generating gas by a gas forming reaction, filling or injecting the dispersion into a mold and allowing it to cure at room temperature. The size of the cells or pores can be controlled by changing pressure within the mold at various pressure differences and various time frames, where higher pressure results in the formation of smaller cells. The size of the cells can also be controlled by changing the temperature of the mold, where higher temperatures result in the formation of larger cells.

FIG. 2 illustrates a cross-sectional top view of a tissue expander 200 according to some embodiments of the present invention. The implant comprises an outer shell 125 comprising at least one stretchable layer made of a deformable resilient polymer, a liquid filled compartment 140, containing, for example, silicone gel or saline, and an inner compartment containing a closed-cell foam element 150 enclosed within, and substantially filling, an inner shell 130. Inner shell 130 comprises a substantially non-stretchable expansion restricting layer. According to this embodiment the foam compartment substantially does not expand and has a fixed surface area due to the expansion restricting layer of inner shell 130. Outer shell 125 made of a resilient deformable polymer may expand according to external pressure exerted to it.

The foam filling of the implants according to embodiments of the present invention is shaped to fit the general shape of the implant. The shell containing the expansion restricting layer 130 is configured to minimize configurational changes of the foam filling, or retain the volume of the foam filling, due to changes of the internal pressure of the gas inside the foam cells, upon changes in the ambient pressure, temperature or both. For example, the expansion restricting layer is configured to prevent an undesired expansion of the foam filling upon a decrease of ambient pressure. The foam filling 150 illustrated in FIG. 2 comprises a single foam element that substantially fills the volume of the implant's inner core, within the liquid-filled compartment.

The outer and inner shells of implants according to embodiments of the present invention may include a plurality of layers. The layers of the outer and inner shells are typically formed of biocompatible, resilient materials, such as silicone, and manufactured by molding. Manufacturing of each shell may be performed by a single-layer molding of each layer independently, followed by joining (for example, gluing) the layers together. Alternatively, over-molding may be performed, where successive layers are molded one on top of the other. Dip molding using pre-formed mandrels can be used for manufacturing each shell, by serial dipping steps to form the layers that constitute the shell. In addition, a combination of the above methods may be used. In some embodiments, the outermost layer of a shell is molded first, and the inner layer(s) are molded over the external layer. The resulting shell is then turned inside out and laid over, e.g., the foam filling, in the case of an internal shell, or over e.g., the liquid-filled compartment, in the case of an external shell. The different components of the implant may be formed of the same material. Alternatively, the different components of the implant may be made of different materials.

Varying thicknesses of the layers that constitute the outer shell and every other layer or structure of the implant according to embodiments of the present invention can be facilitated by transfer/compression/injection molding or any other technique using molds for manufacturing.

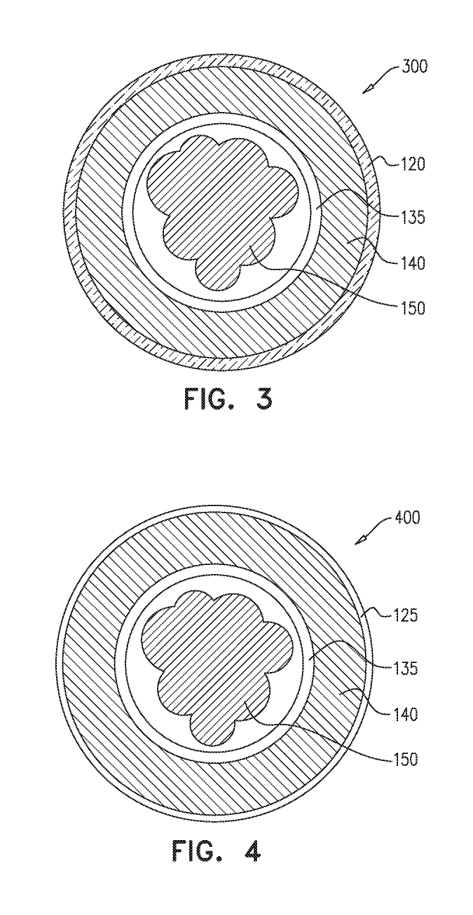

FIG. 3 illustrates a cross-sectional top view of a tissue expander 300 according to some embodiments of the present invention, suitable, for example, for breast augmentation and/or reconstruction. FIG. 3 shows a tissue expander 300 that comprises an external shell 120 reinforced with an expansion restricting layer defining a fixed surface area of the implant. The inner core comprising a closed-cell foam filling 150 (e.g., silicon foam filling) is contained within an inner shell 135 that is stretchable and made of a resilient deformable polymer. In the illustrated embodiment inner shell 135 has no expansion restricting layer. Tissue expander 300 further comprises a liquid compartment 140 surrounding foam filling 150. The expansion of the closed-cell silicone foam in the illustrated embodiment is restricted by liquid compartment 140 and the expansion restricting layer embedded in external shell 120.

FIG. 4 illustrates a cross-sectional top view of a tissue expander 400 according to additional embodiments, suitable, for example, for breast augmentation and/or reconstruction. Tissue expander 400 of FIG. 4 includes a stretchable external shell 125 and a stretchable internal shell 135. The inner core comprises a closed-cell silicone foam filling 150 surrounded by a liquid-filled compartment 140. The expansion of closed-cell silicone foam filling 150 is partially restricted by the elastic forces of the polymers forming inner shell 135 and external shell 125, and by liquid-filled compartment 140. In the illustrated embodiment, external shell 125 and internal shell 135 may stretch such that the 3-D configuration of the tissue expander is changed, in response to external pressures applied to it. In other embodiments, the closed-cell foam filling may be formed of a polymer(s) that substantially does not expand (as defined above for the expansion-restricting layer).

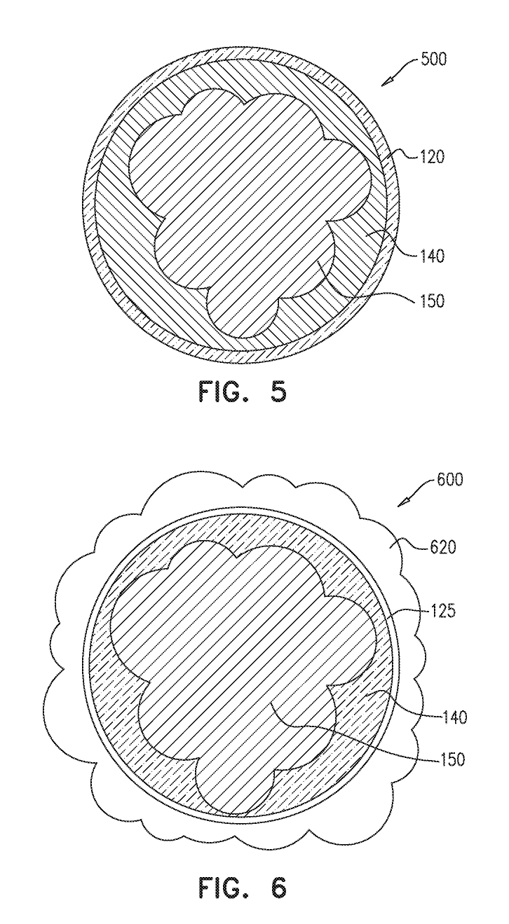

FIG. 5 illustrates a cross-sectional top view of a tissue expander 500 according to some embodiments of the present invention, suitable, for example, for breast augmentation and/or reconstruction. Tissue expander 500 of FIG. 5 includes an external shell 120 reinforced with an expansion restricting layer defining a fixed surface area of the tissue expander. The inner core comprises closed-cell foam filling 150 (e.g., a silicone foam filling) floating freely in a liquid-filled compartment 140. The expansion of the closed-cell silicone foam is partially restricted by liquid-filled compartment 140 and is also restricted by the expansion restricting layer embedded in the external shell 120.

FIG. 6 illustrates a cross-sectional top view of a tissue expander 600 according to some embodiments of the present invention, suitable, for example, for breast augmentation and/or reconstruction. Tissue expander 600 illustrated in FIG. 6 comprises a shell 125 that is preferably reinforced with an expansion restricting layer defining a fixed surface area of the tissue expander. The inner core of tissue expander 600 comprises a closed-cell foam filling 150 floating freely in a liquid compartment 140. The expansion of the closed-cell foam filling is partially restricted by liquid compartment 140 and also by the expansion-restricting layer of shell 125. Tissue expander 600 further comprises a layer of closed-cell silicone foam 620 attached to the external surface of the tissue expander, to enhance the softness of the tissue expander and improve tactile feedback thereof upon touching.

Tissue expanders according to embodiments of the present invention may include a code identifier (label) on their surface or embedded therein, for non-invasively identifying the implants after they are implanted. According to one embodiment the code identifier may be printed during the manufacturing process. “Ink” materials may include, e.g., polymers, which can be distinguished and detected by optical, electro-optical, electromagnetic, ultrasonic detection means, or other detection means known in the art. For example, a printing material may include a radio-opaque material like barium sulfate, may contain gas bubbles to be detected by ultrasound, may contain a color different from the color of the implant layer on which it is printed or in which it is embedded to be detected optically, may contain magnetic material defining a different magnetic imprint for each code, or any combination of the above, but not limited to the above. According to another embodiment, the code may be cut by laser or any mechanical cutting device or method from a sheath made of the above described “ink” materials. The code may be generated by a computer, either randomly or according to a pre-determined algorithm. The code may be a printable character or any drawing or shape. The code may be saved to a computerized database to be retrieved when needed through a local network or over the web. The code can advantageously be retrieved non-invasively from the implant while the implant is still implanted in the patient, without the need to extract the implant by surgery. Once the code is retrieved, some or all information regarding the implant, the patient and the operating physicians can be retrieved over the web or in any other method as allowed by authorities. In preferred embodiments, the code identifier is made of a material having mechanical properties similar to those of the shell it is embedded in or attached thereto, thus not affecting the mechanical properties of the shell. If the code identifier is embedded in, or attached to, an outer shell, the code identifier preferably has the same tactility of the shell, thus being impalpable to a subject touching the implant from outside the body. The code identifier may be placed within any compartment of the implant. The code identified may be partially embedded in any shell of the implant and partially protruding from the shell. The code identifier is typically made of a material having mechanical properties that do not damage the integrity of any shell or other component of implant.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A human implantable tissue expander comprising:
an inner foam filling;
a liquid-filled compartment enclosing said inner foam filling; and
a shell comprising a substantially non-stretchable resilient expansion restricting layer configured to retain a fixed surface area of said foam filling upon changes of ambient pressure, temperature or both,
wherein said inner foam filling constitutes at least 60% of the total volume of said tissue expander.

2. The tissue expander of claim 1, wherein said inner foam filling constitutes at least 75% of the total volume of said tissue expander.

3. The tissue expander of claim 1, wherein said inner foam filling constitutes at least 95% of the total volume of said tissue expander.

4. The tissue expander of claim 1, wherein said inner foam filling comprises a single foam element.

5. The tissue expander of claim 1, wherein said inner foam filling comprises at least two foam elements.

6. The tissue expander of claim 1, wherein said inner foam filling comprises closed-cell foam.

7. The tissue expander of claim 6, wherein said closed-cell foam is silicone foam.

8. The tissue expander of claim 6, wherein said closed-cell foam comprises closed-cell foam beads.

9. The tissue expander of claim 1, wherein said liquid is silicone gel.

10. The tissue expander of claim 1, wherein said liquid is saline.

11. The tissue expander of claim 1, wherein said shell comprising a substantially non-stretchable expansion restricting layer is an outer shell enclosing said inner foam filling and said liquid-filled compartment.

12. The tissue expander of claim 11, further comprising an additional shell comprising a substantially non-stretchable expansion restricting layer, wherein said additional shell is an inner shell between the liquid-filled compartment and the inner foam filling, enclosing the inner foam filling.

13. The tissue expander of claim 1, further comprising a shell comprising a stretchable layer formed of a resilient material.

14. The tissue expander of claim 13, wherein said shell comprising a stretchable layer formed of a resilient material is an outer shell enclosing said inner foam filling and said liquid-filled compartment, and wherein said shell comprising a substantially non-stretchable resilient expansion restricting layer is an inner shell between the liquid-filled compartment and the inner foam filling, enclosing the inner foam filling.

15. The tissue expander of claim 13, wherein said shell comprising a stretchable layer formed of a resilient material is an inner shell between the liquid-filled compartment and the inner foam filling, enclosing the inner foam filling, and wherein said shell comprising a substantially non-stretchable resilient expansion restricting layer is an outer shell enclosing said inner foam filling and said liquid-filled compartment.

16. The tissue expander of claim 1, further comprising an outer layer of closed-cell foam.

17. The tissue expander of claim 1, further comprising a code identifier on a surface thereof or embedded therein, for non-invasively identifying said tissue expander when implanted in a subject.

18. The tissue expander of claim 17, wherein said code identifier is embedded in, or attached to, a shell of said tissue expander, and made of a material having mechanical properties which are similar to the mechanical properties of said shell.

19. The tissue expander of claim 17, wherein said code identified is partially embedded within a shell of said tissue expander.
